(19) United States
(12) Patent Application Publication
(10) Pub. No.: US 2005/0171399 A1
(43) Pub. Date: Aug. 4, 2005

(54) TR309 - portable otoscope video viewer

(76) Inventors: Tony C. Rich, Gainesville, FL (US); Diane B. Rich, Gainesville, FL (US)

Correspondence Address: TONY C. RICH, MD, 1710 N.W. 42ND AVENUE, Gainesville, FL, 32605, US

(21) Appl. No.: 11/037,885

(22) Filed: Jan. 18, 2005

Related U.S. Application Data
(60) Provisional application No. 60/539,019, filed on Jan. 23, 2004.

(51) Int. Cl. A61B 1/04; A61B 1/267

(52) U.S. Cl. 600/112; 600/200

(57) ABSTRACT

The TR 309 is a portable otoscope video adaptor and viewing unit, designed to make the examination of ears, nose, throat, and eyes more easily attainable and to increase the size of the obtained view. It contains a built-in Camera/LCD/video recorder/otic thermometer in a handheld unit which fits the Welch Allyn or Heine otoscope or works totally independently. It is designed to fit into any shirt pocket for portability. It also can be easily pushed to the side, allowing the normal use of the Welch Allyn or Heine otoscope, or the 309's otic adapter, allowing foreign body removal or tympanocentesis. The TR 309 also provides for an unobstructed view and access to the eardrum, nose, throat, or eye for surgery while under real time view on the unit's LCD. A new ear specula and needle specifically designed for tympanocentesis is also described.

### CROSS-REFERENCE TO RELATED APPLICATIONS

This application claims the benefits of Provisional Patent No. 60/539,019, filing date Jan. 23, 2004, title: TR 309—Portable Otoscope Video Viewer, granted to Tony Curtis and Diane Cross Reference to other related patents:

| | | | |
|---|---|---|---|
| 1515771 | November 1924 | Greenwald | 128/9 |
| 1693021 | November 1928 | Cameron | 128/9 |
| 2154885 | April 1939 | Speelman | 128/9 |
| 3728998 | April 1973 | Heine | 128/9 |
| 3934578 | January 1976 | Heine | 600/200 |
| 4006738 | February 1977 | Moore, et al. | 128/9 |
| 4147163 | April 1979 | Neuman, et al. | 600/200 |
| 4366811 | January 1983 | Riester | 128/9 |
| 4685452 | August 1987 | Riester | 600/200 |
| 5239984 | August 1993 | Cane, et al. | 600/112 |
| 5345926 | September 1994 | Chikama | 600/200 |
| 5363839 | November 1994 | Lankford | 600/112 |
| 5527261 | June 1996 | Monroe | 600/200 |
| 5919130 | July 1999 | Monroe, et al. | 600/200 |
| 6186944 | February 2001 | Tsai | 600/200 |
| 6272375 | August 2001 | Katzip, et al | 600/474 |
| 6361489 | March 2002 | Tsai | 600/109 |
| 6383133 | May 2002 | Jones | 600/200 |
| 6511420 | January 2003 | Farell, et al | 600/167 |
| 6704053B1 | March 2004 | Masahito, et al | 348/340 |

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

For many years physicians have had to rely on the Welch Allyn, Heine, or other similar otoscopes to determine the normality or abnormality of his patient's eardrums with their inherent limitations. These limitations include but are not limited to: Small image size, difficulty in obtaining an adequate view of the tympanic membrane, lack of real time viewing while doing procedures to the eardrum, squirming uncooperative patients, and more. Also, these have frequently required the physician to maintain a prolonged bending of his back to obtain an adequate view, leading in some cases to the physician's early and chronic back pain. In addition these units have been cumbersome and required a large attachable battery source or wired attachment to an A/C adaptor.

The diagnosis (or misdiagnosis) of otitis media has often had tremendous impact on the individual patient, ranging from the prescription of unnecessary antibiotics, unnecessary antibiotics, unnecessary blood or other medical test, or to the requirement for return visits to the physician to diagnose ear infections which were missed on the first visit. These prior devices also provided no means to capture and fix a view of the eardrum and canal for prolonged review, analysis, training, and/or second opinion in a portable, practical, and affordable means. It is also noted, the Welch Allyn and Heine type otoscopes are certainly the most prevalent such devices currently in use, most physicians trained with them and are comfortable with their use, and most medical facilities have a substantial financial investment in these type instruments.

It has also not been easy in the past to combine a portable otoscope head which provided a view of the eardrum and its reaction to an impact of inrushing air (pneumonoscopy) and simultaneously view this on a LCD or video screen. The TR 309 is designed to overcome these limitations.

Tympanocentesis using the afore mentioned products has been extremely difficult, so much so that in actual practice it has been often foregone altogether, even though this is often the best treatment option for the patient for recurrent, persistent ear infections. This has led to the lack of the ability to test for antibiotic susceptibility of the infecting agent, and the empiric choice of antibiotic by the physician with the resulting increasing antibiotic resistance of many bacteria today. The TR 309 Portable Otoscope Video Adapter and Viewer and it's newly designed ear specula and needle aspirator are designed to alleviate all these problems and to provide a better view of the examination of the ear, eyes, and nasal passages and/or oropharynx.

### BRIEF SUMMARY OF THE INVENTION

The TR 309 Portable Otoscope Video Adapter and Viewer is designed to make the medical examination of patient's ears, eyes, nose, and throat more easily attainable and to also improve the accuracy of said exam. Its use is so easy any nurse or office assistant can be trained to use it, obtaining both the patients temperature and a recorded view of each eardrum in a minimum amount of time. This will frequently free up the physicians time for more important matters and also save him from the risk of degenerative back disease. The TR 309 contains a built-in camera/LCD/video/otic thermometer recorder in a hard-shell compact plastic case which fits solidly onto the Welch Allyn or Heine type otoscope heads and is designed to fit into any physician's shirt or lab pocket for true portability. With its supplied adaptors the TR 309 has basically all the same components and functions as when coupled with a Welsh Allyn or Heine otoscope head, and can function as a stand alone unit. Both configurations are designed to provide the physician or his assistant with an actual real time view of the ear canal, eyes, eardrum, pneumonoscopy, nasal passages, or oropharynx, through a small video camera and a viewing LCD built into its body.

This also offers advantages over similar devices in that the video unit is easily pushed to the side or removed totally, allowing the normal use of the Welsh Allyn or Heine otoscope or the TR 309's own otic adapter, allowing ear wax or foreign body removal. Tympanocentesis can be done through the body of the otoscope head as is currently practiced by thousands of physicians, or by using the newly designed otoscope speculum and needle which makes the procedure much easier, quicker, and safer.

The TR 309 also allows the physician or his nurse to “freeze frame” a view at any point during the exam to get the best possible view for each eardrum and allows for prolonged viewing, study, or consultation.

The TR 309s built-in adaptor fits any Welch Allyn or Heine otoscope head, whether portable or wall mounted. It is powered by its own built-in NiCad battery which can be recharged with the supplied battery charger. The TR 309 also incorporates all these features into a single hand held adaptable unit which provides all of the above features plus a functioning tympanic thermometer with the use of the supplied adaptors. In short, the TR 309 will revolutionize the diagnosis of ear infections and other afflictions and greatly simplify the practice of medicine.

With it's attachable newly developed otoscope speculum which has an oblique opening slit built into it's side, the physician is able to do a tympanocentesis or other surgical procedure through said slit with a newly developed modified needle (FIG. 13), while simultaneously viewing the patients eardrum on the TR 309s LCD, thus providing for a more accurate and safer procedure than in the past. These specula are disposable, sterile, and thus provide for ease of maintaining sterility in these type procedures.

The TR 309 can be used as a teaching tool in University settings, but is just as practical and affordable for any physician's office. It can be carried easily in a lab jacket or coat pocket in its soft leather/felt carrying case.

### BRIEF DESCRIPTION OF THE SEVERAL VIEWS OF THE DRAWINGS

FIG. 1—REAR VIEW—TR 309. This shows the rear of the TR 309(which is app. 2 by 2 inches by 1 inch in size) and shows the LCD which is approximately one and one-half by one and one-half inches and is fixed into position on the backside of the frame of the TR 309. Button #1 turns the unit on or off, including LCD viewing. Button 2 starts or restarts the video camera with LCD viewing, for a maximum period of 10 seconds for each ear (held in on board temporary memory). Button 3 “freeze frames” a view (for the right ear), which is held in memory until actively erased by the user. Button 4 does the same thing for a second view for the left ear. Button 5 functions much the same as the “rewind” button on a VCR enabling the viewer to review the recording. Button 6 clears the memory bank for the left or right view, or push twice to clear the entire memory. Choice of left or right labels is enabled by internal software which is self-instructive to the user. Button 7 causes the temperature of the eardrum to be shown on the LCD (thermometry). Button 8 turns on or off the internal light source of the TR 309 for use in its stand alone mode.

FIG. 2A—FRONT VIEW—TR 309. Shows the Front View of the TR 309 and illustrates the adaptor (#9) which secures the TR 309 to the Welch Allyn Otoscope head. It is approximately 1 inch by 3/8 inch and slides easily into the standard fitting for the lens viewfinder of the standard Welch Allyn Otoscope head. This provides a firm and stable mounting at a well functioning angle for examining the patient's eardrum for the TR 309. #10 is a high quality close-up photography lens which provides the correct focal length for viewing the patient's eardrum. It's focal length can be adjusted by the Fine Focus Adjustment wheel (Fig., item #13) to accommodate the varying sizes of the patient's ear canal or it can be shifted out of the way for the use of a different attachment such as the Ophthalmoscope Adaptor (FIG. 10, item 32). The self contained light source (#11) can be turned on with #8, FIG. 1 when the 309 is used as a stand alone unit or turned off when used with a Welsh Allyn or Heine otoscope.

FIG. 2B—FRONT VIEW—TR 309. Shows the Heine type otoscope head Adapter Ring (#12). These spring loaded clamps fit the TR 309 securely onto a Heine type otoscope head. They slide up or down in the slits indicated to clamp onto the Heine Otoscope head. #10 is the same lens as referenced in the preceding paragraph. #11 is the same internal light source as in FIG. 1.

FIG. 3—LEFT SIDE VIEW—TR 309 WITH WELCH ALLYN OTOSCOPE HEAD. This shows the typical Welch Allyn Otoscope head (#17) along with the Left Sided View of the TR 309 (#15), and how the connection is made via the adapter ring (#9). In this view the Welch Allyn Viewing Lens is removed and this shows how the Adaptor fitting (#9) for the TR 309 fits into the receptacle for the Welch Allyn Viewing Lens fitting to form a secure well-fitting adaptor for the TR 309. #19 shows the standard Welch Allyn Power Handle containing its usual rechargeable battery. The TR 309 is powered by a built-in independent battery supply which can be recharged by #20 (Battery Charger) and connected to the recharger via the plug-in for the recharger (#16). #10 shows the close up photo lens. #11 shows the internal light source. #13 shows the Fine Focus Adjustment Ring. #14 shows the built in LCD. Item 18 shows the regular air connection hole for the Welsh Allyn Otoscope head.

FIG. 4—BATTERY CHARGER. Standard battery charger with plug-in adapter which fits into #16, FIG. 3.

FIG. 5—FRONTAL OBLIQUE VIEW OF THE TR 309. This is a left frontal oblique view of the TR 309 showing the Adapter Ring (#9) in slightly more detail. #10 is the viewing lens. #11 is the internal light source as already referenced.

FIG. 6—BACK OBLIQUE VIEW OF THE TR 309. This is a 3D view of the back of the TR 309. It shows the LCD screen and items 1 to 6 are the same as described in FIG. 1. Item 6a shows the USB connector socket for connection to a computer if desired, but this is not necessary for regular operation of the TR 309.

FIG. 7—BOTTOM OF TR 309. This view shows the bottom of the TR 309 and its approximate bottom dimensions, one inch by two inches. It also shows the screw-in adapter (#21) for the attachment of the TR 309 Battery Handle (FIG. 8). It also shows the adaptor ring (#9) for attachment to the Welsh Allyn Otoscope Head.

FIG. 8—BATTERY HANDLE W/SCREW ON ADAPTOR FOR THE TR 309. This shows the battery handle extension which can be screwed into the bottom socket of the TR 309 via #22 to provide extra battery power via a rechargeable and replaceable NiCad Battery (23). Battery access and removal is by a screw-on cap (#25). The battery handle provides for better stability and ease of use in the TR 309's stand alone mode. The battery handle is approximately 5 inches long and one and one-quarter inches in diameter.

FIG. 9—OTIC ADAPTOR. This view shows the otic adaptor (#27) which incorporates a modified ear specula adaptor (#26). This fits onto the Adaptor for the Welch Allyn Otoscope head on the TR 309 to enable the physician to use all the features of the TR 309 without the need for a Welch Allyn device at all. By attaching standard ear specula to #26 while it's attached to the TR 309, the physician is able to obtain the same views as with a Welsh Allyn or Heine otoscope unit. He is also able to obtain a physical seal of the ear canal enabling pneumonoscopy to be done using only the TR 309 and an attachable standard air tube and insufflator via #28.

FIG. 10—OPHTHALMOSCOPE ADAPTOR. This shows the ophthalmoscope unit (#32) which attaches to the TR 309 and functions much like any other standard ophthalmoscope. #29 shows the number of the ophthalmoscope lens currently in place. #30 shows the aperture for the lens and through which the TR 309 focuses on the retina. #31 shows the adaptor ring which attaches the unit to the TR 309.

FIG. 11—THE TR 309, WITH ATTACHMENTS. This view shows the TR 309 with all its main attachments attached, such as the Otic Adaptor (#27). #10 shows the lens unit for the TR 309. #13 shows the fine focus knob. #14 shows the LCD. #16 shows the connector for the battery recharger. #22 shows the screw on adaptor for the battery holder. #23 shows the rechargeable NiCad battery. #25 shows the screw on end cap for the battery holder. The Ophthalmoscope unit is not shown in this view but attaches in the same way and place as the Otic Adaptor (#27).

FIG. 12—MODIFIED EAR SPECULA. This shows a modified ear speculum (#33) which has a slit (#34) preformed in it for insertion of the K-Needle in order to provide for easy tympanocentesis.

FIG. 13—K-NEEDLE. This shows an elongated aspiration needle with an approximate 25 degree angle bend at approximately 3/4 inch from it's hub to facilitate tympanocentesis while under view on the TR 309 and using the modified ear specula (FIG. 12, #33). There is approximately ¾ inches of needle after the bend to better facilitate the procedure. It is shown attached to a standard TB syringe in FIG. 13.

### DETAILED DESCRIPTION OF THE INVENTION

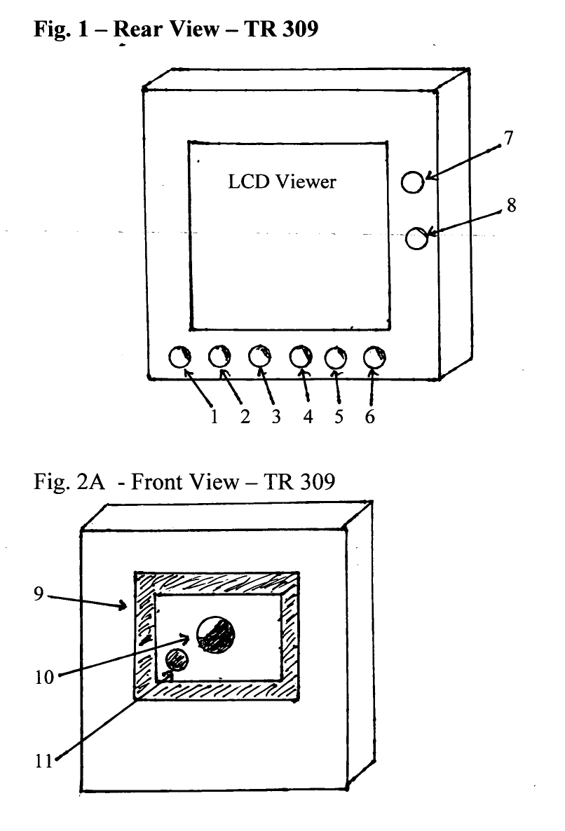

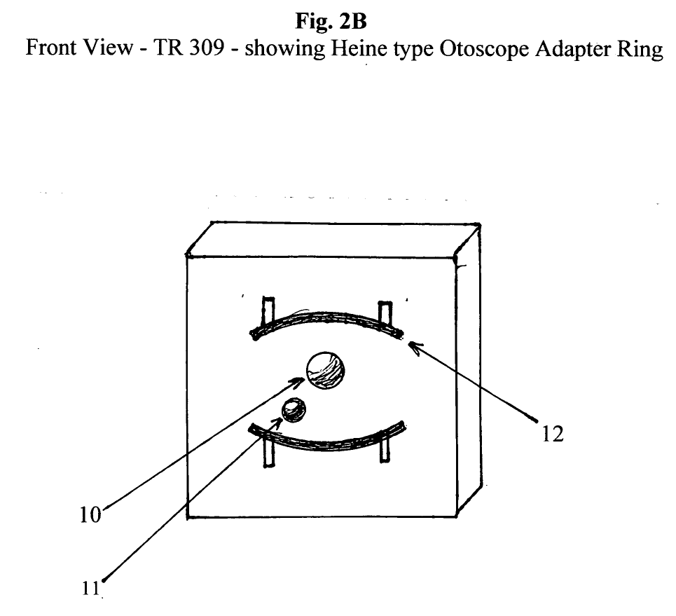

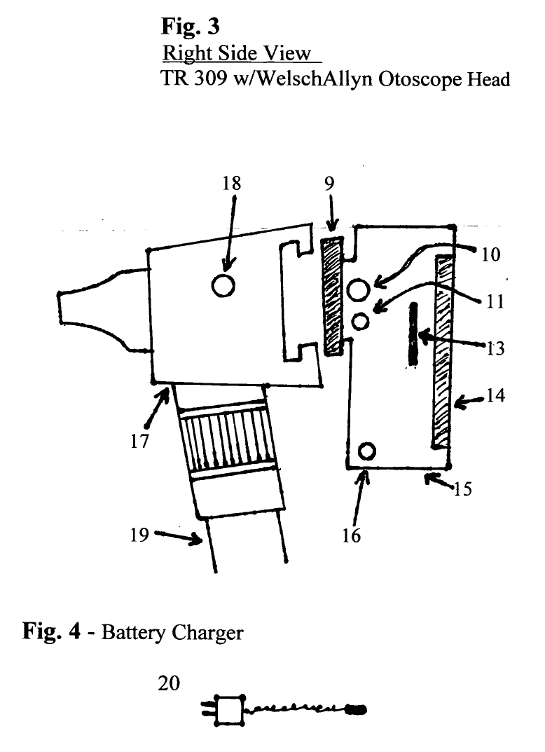

The TR 309 Portable Otoscope Video Viewer contains a built-in Camera/LCD/Video Recorder (FIGS. 1, 3, 5, 6 and 11) in a molded plastic composite hard-shell compact case. The camera, LCD, and video recorder use pre-existing technology. The TR 309 is approximately 2 inches by 2 inches. The LCD is approximately 1½ by 1½ inches. It fits solidly onto the Welch Allyn Otoscope head (FIG. 3, # 17) via an adaptor (FIGS. 3 & 9), which is made of hardened rubber for ease of fit. The TR 309 is designed to fit into any physician's coat or shirt pocket. It can also be adapted to a Heine type otoscope using the spring loaded adapters shown in FIG. 2B. #12. These are pulled by spring pressure to securely fit it to the otoscope head. These (#12) are also made of hardened rubber.

The LCD and micro-camera technology is pre-existent and is as compact as possible, although adapted for this particular application. An example of a micro-camera is referenced in Patent No. 6704053 B1. The TR 309 incorporates this technology for true small size and portability to allow the physician or his assistant to take it wherever he needs it and to adapt it to his available otoscopes, or to function independently with it's own adaptors (FIGS. 8, 9, and 10). Of course the lens of the camera is changed to allow for focus on the eardrum or on the retina with the ophthalmoscope. Camera Controls include (FIG. 1, items 1-8):
#1—On/Off power Switch
#2—Start/Restart (Show)—starts LCD recording to memory storage (10 second memory each for right and left ear). Push twice to view the recorded sequence.
#3—Freeze Frame—Push once to freeze a right ear view and push twice to view the picture in memory.
#4—Freeze Frame—Push once to freeze a left ear view and push twice to view the picture in memory.
#5—Rewinds the recording(s) to the beginning. First Push #3 to choose the right ear view or push #4 to choose the left ear view.
#6—Clears all memory. First push #3 to choose the right ear or #4 to choose the left ear views.
#7—Causes the temperature of the eardrum to be shown on the LCD (thermometry).
#8—Button 8 turns on/off the internal light source of the TR 309.

The built-in software enables the user to follow the directions on the LCD to record 10 second views of the left and right ear and to “freeze frame” a view of each eardrum. These remain in memory until cleared by #6 (Clear Memory Button). Views of the left and right ear can be recalled by pressing #s 2, 3, or 4 twice, depending on the view one wishes to see.

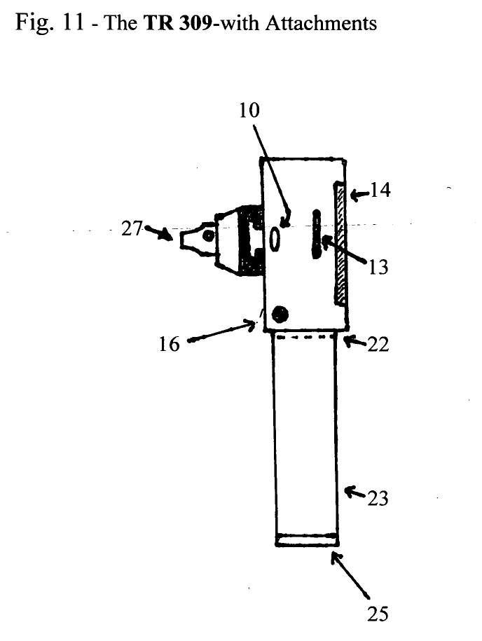

The TR 309 can also be used as a stand alone unit (as shown in FIG. 11 using the adaptors shown in FIGS. 8, 9, and 10. Both adaptations are designed to provide the physician with an actual time view of the ear canal and eardrum through a small video camera (FIG. 1 and FIG. 2A) built into the body of the TR 309. This camera provides a video image of what the physician would see looking through a normal otoscope view, but magnifies the image size greatly (by a factor of approximately ten) which is then displayed on the LCD which is built into the back of the TR 309 using existing technology. Control buttons (#s 1-8, FIG. 1) are also made using standard pre-existing technology, and are each covered by a protective rubber coating to keep out dirt and dust.

The focus of the Close-up Lens (FIG. 3 #10) can be finely adjusted by use of the fine focus adjustment wheel (FIG. 3, #13). The TR 309 also records 10 seconds of viewing time for each ear or eye in its built-in memory, which is available for review at the physician's convenience and/or downloading into a computer via a standard USB type 2 connector (#6a, FIG. 6). It also provides for the recording and viewing of ear drum movement under air pressure (pneumoscopy) using an add on standard air insufflator bulb which can be connected to the air hole on the standard Welsh Allyn type otoscope (FIG. 3, #18) or onto (FIG. 9, #28), using the same procedure which is well described in the medical literature.

The TR 309 also allows the physician or his nurse to “freeze frame” (FIG. 1, #3) a particular view at any point during his exam to get the best view possible for close inspection of each eardrum. This solves many of the drawbacks to the brief glance of the eardrum and canal obtained with a normal otoscope head in that the physician can examine the view of the eardrum and ear canal as long as he needs to without requiring the patience or cooperation of the patient, and he can also obtain a second opinion, or use it for teaching purposes to other physicians, or to show to the patient or the patient himself or his parents.

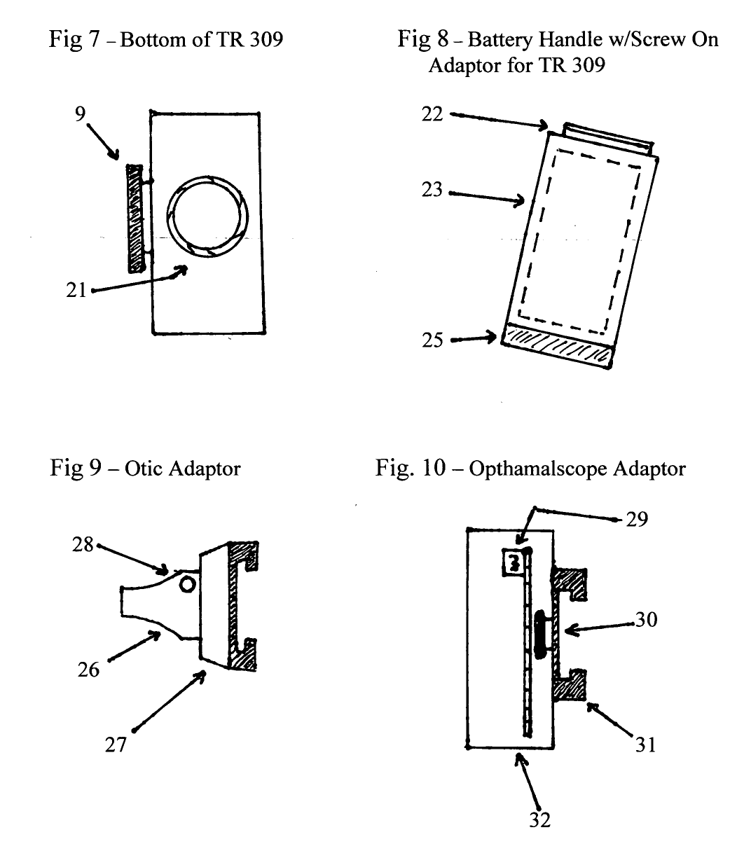

TR 309's built-in adaptor (FIGS. 2A, 3, and 5) fits any Welch Allyn otoscope head, whether portable or wall mounted. With the Heine type adapter (FIG. 2B) all functions are also capable of being done with a Heine type otoscope. The light source for the otoscope is from the Welch otoscope or Heine Otoscope in those adaptor modes. In its stand alone mode it is powered by its internal battery (Ni-Cad Rechargeable) and these can be augmented by its Battery Handle (FIG. 8) which holds an additional NiCad rechargeable battery similar to the one in portable Welsh Allyn Otoscope handles. This provides more and longer lasting power, and attaches via a screw-on adaptor (FIGS. 7, 8, and 11) and it's battery can also be recharged via the supplied recharger through the plug-in connector on the body of the TR 309 (FIG. 3, #16). The built-in light source can be turned on or off with it's built-in switch (FIG. 1, #8).

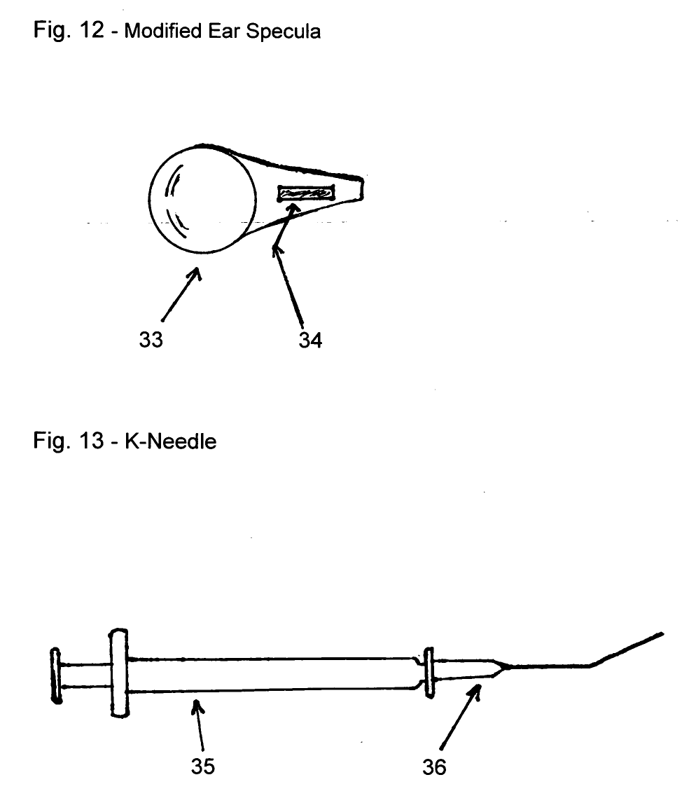

The TR 309's own Otic Unit with Adaptor is shown in FIG. 9 and can use standard ear specula or the newly designed specula shown in FIG. 12, #33. In FIG. 9, #28 and #26 is made from formed aluminum alloy and the adapter (#27) for attaching it to the TR 309 is made from hard formed rubber to allow for variations in fit. #28 is a preformed alloy fitting for attaching an air tube and rubber bulb for pneumoscopy.

The Ophthalmoscope attachment (FIG. 10, #32) for the 309 is shown in FIG. 10 and is made of a molded plastic body into which a rotating lens wheel which contains the usual assortment of viewing lens as in other ophthalmoscope units is mounted (pre-existing technology). #29 shows the lens number in use by mechanical means and #30 shows the aperture for viewing and its light source and are arranged as in most ophthalmoscopes. The adaptor (#31) to connect it to the TR 309 is made of hard formed rubber to allow for slight variations in fit. The Ophthalmoscope attachment provides all the normal views obtainable with a standard ophthalmoscope and the TR 309 magnifies them for view on its LCD. These also can be stored in memory for later viewing, freeze-framing, or downloading into a computer via the 309's USB 2 connector (FIG. 6, #6a).

FIG. 8 shows the battery handle (metal alloy) with enclosed rechargeable Ni-Cad battery (#23) which is similar to the Welsh Allyn battery handle and battery and could be designed to simplify supply problems. It should be approximately five inches long and 1 and ½ inches in diameter. @22 shows the metal screw type adaptor for attaching it to the bottom of the TR 309 via #21, FIG. 7. #25 shows the screw-on cap which allows access to the battery compartment.

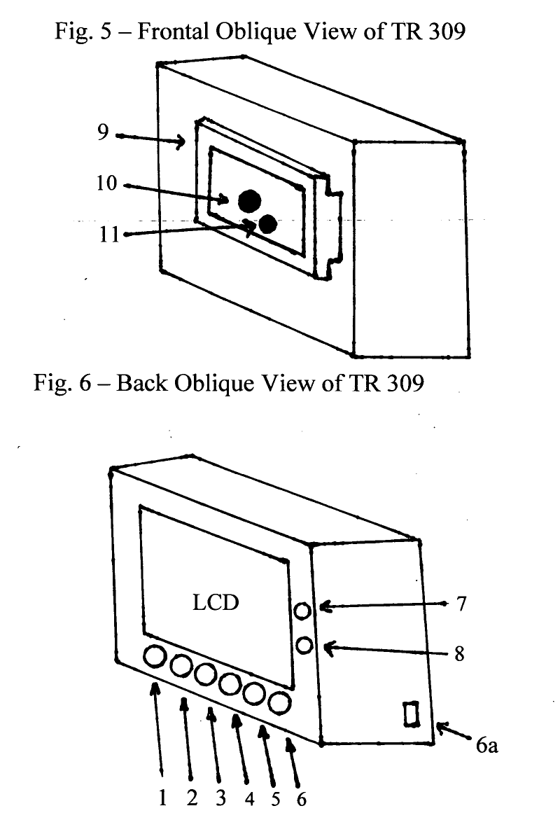

FIG. 11 shows the TR 309 assembled in its stand-alone configuration with its attachment's attached. In this mode it can completely replace a Welsh Allyn or Heine type otoscope and furthermore provides an on-unit LCD (#14) for a much larger and detailed image of the eardrum (or retina, or other object being viewed, along with the previously described options. #13 shows the fine focus wheel. #10 shows the camera lens and aperture unit (pre-existing technology). The Otic Adaptor just slides into place via #27, FIG. 9 and #9, FIG. 5. #16 shows the plug-in for the battery charger (#20, FIG. 4). The battery handle screws into the bottom of the TR 309 via #22, FIG. 8, and #21, FIG. 7.

FIG. 12 shows the modified ear specula (#33) which are of molded plastic with a preformed slit (#34). It is approximately the same size as a #4 standard otoscope speculum and the slit is approximately 1/4 inch wide and 1/2 inch long. Manufacture is the same as existing ear specula except for the slit which is a simple cut-out. This allows for easy insertion and manipulation of the K-Needle (FIG. 13, #36) for performance of tympanocentesis or other surgical procedures with live viewing on the LCD of the TR 309.

FIG. 13 shows the K-Needle which is manufactured as existing needles are except that it has a 25 degree angle bend approximately ¾ inches down its barrel and it extends ½ inch beyond this bend. This allows for easy insertion and manipulation through the slit shown in FIG. 12, #34 in order to perform tympanocentesis while viewing the procedure on the TR 309's LCD. #35 shows a standard TB syringe with #36, the K-Needle, attached, with its plastic hub and surgical steel needle which allows for puncture of the eardrum and aspiration of the inner ear fluid for bacterial culture.

In short, the TR 309 will bring the diagnosis and treatment of ear infection and opthalmoscopy into the 21^{st} century. It can enable the office nurse or assistant to obtain inner ear views while she is also obtaining the patients temperature, thus freeing up more time for the physician to spend on other matters, without compromising the quality of medical treatment. It also can be used as a teaching tool in University settings, but is just as practical and affordable for any physician's shirt pocket and slips easily in and out of a soft leather/felt carrying case.

1. The use of a portable, handheld, combination of a video camera, an LCD, alone or in combination, and it's programming to enable the viewer to obtain recorded video and/or single frame views of the ear, ear canal, eardrums, nasal passages, oropharynx, and/or ophthalmoscope views of the eye, it lens, and/or the retina.

2. The use of portable hand-held technology to store any/or all of the above obtained views (claim #1) in it's memory and to be viewed later on the same or different hand-held unit or downloaded into a computer for display on any type screen such as a LCD, projection unit, TV, etc.

3. The use of a portable hand-held unit which provides real time views of the eardrum or canal, and/or pneumoscopy which is displayed on a LCD built into the unit and can be stored in memory for later viewing and also does any or all of the above three claims in the same unit.

4. The use of a portable hand-held unit which performs any or all of the above three claims and can attach to and utilize the Welch Allyn or Heine type otoscope head as a viewer, and/or light source.

5. The use of a portable handheld unit which allows a video view of the desired ear, nose, throat and/or the eyeball and retina views and which allows, at the same time, surgical procedures to be done on the same while actively viewing the procedure on real-time live video.

6. The design, use, and method of a newly designed otoscope speculum tip with a slit which allows for the performance of tympanocentesis or other ear surgery while viewed in real time on the TR 309's LCD or another video monitor.

7. The design and shape of a specially shaped needle (the K-Needle), which can be used to access surgical sites via the described otoscope speculum in claim 6 and it's use thereof in the herein described method of tympanocentesis.
